(19) United States
(12) Patent Application Publication
(10) Pub. No.: US 2025/0130201 A1
(43) Pub. Date: Apr. 24, 2025

(54) Ion Analyzer and Ion Analyzing Method

(71) Applicant: SHIMADZU CORPORATION, Kyoto-shi (JP)

(72) Inventors: Yuta MIYAZAKI, Kyoto-shi (JP); Hidenori TAKAHASHI, Kyoto-shi (JP)

(21) Appl. No.: 18/921,189

(22) Filed: Oct. 21, 2024

(30) Foreign Application Priority Data
Oct. 23, 2023 (JP) 2023-181595

(51) Int. Cl. G01N 27/68; G01N 33/00

(52) U.S. Cl. CPC G01N 27/68; G01N 33/0027

(57) ABSTRACT

In an ion analyzer dissociating an ion originating from a sample by bringing a radical into contact with the ion within a reaction chamber, a source gas from which the radical is to be generated is introduced into a generation chamber. A power supplier supplies electric power for generating an electric discharge within the generation chamber. A source-gas supplier supplies the source gas to the generation chamber. A controller controls the source-gas supplier and the power supplier to perform, in a switchable manner, a radical introduction mode where the electric power is supplied to the generation chamber while the source gas is supplied to the generation chamber for supplying the radical to the reaction chamber, and a source-gas-only introduction mode where the electric power is not supplied to the generation chamber while the source gas is supplied to the generation chamber for supplying the source gas to the reaction chamber.

### TECHNICAL FIELD

The present invention relates to an ion analyzer and an ion analyzing method for analyzing an ion originating from a target component contained in a sample.

### BACKGROUND ART

In order to identify a sample component (e.g., a high-molecular compound) contained in a sample and analyze its structure, the technique of mass spectrometry has been widely used in which an ion having a specific mass-to-charge ratio (m/z) is selected from ions originating from the sample component, and various product ions generated by dissociating that ion are detected after being separated from each other according to their mass-to-charge ratios. Various methods have been known as techniques for dissociating ions. One of those known techniques is a radical-induced dissociation method in which various kinds of radicals are made to attach to or react with an ion to dissociate this ion (although this technique may also be called the “radical attachment dissociation method”, the term “radical-induced dissociation method” is used in the present description).

For example, Patent Literature 1 discloses a method in which radical-induced dissociation of an ion is caused by irradiating the ion with hydroxyl (OH) radical, oxygen radical, nitrogen radical or other kinds of radicals generated by radio-frequency electric discharge, and the thereby generated product ions are subjected to mass spectrometry. Patent Literature 2 discloses that the location of a double bond which determines the bioactivity of a lipid can be analyzed by an MS/MS analysis employing a radical-induced dissociation method which uses hydroxyl and oxygen radicals.

Patent Literature 3 discloses one example of a radical generation device for generating radicals to be used in the previously described radical-induced dissociation method. This radical generation device has a helical antenna including a quartz tube with a strip of conductor wound around its outer circumferential surface. Source gas, such as steam, is introduced into the quartz tube, and radiofrequency (microwave) power is supplied to the helical antenna to generate a cloud of plasma within the quartz tube and create radicals within this plasma. A magnet generating a strong magnetic field is placed on the outside of the quartz tube. This magnetic field induces electron cyclotron resonance, which increases the density of the plasma within the quartz tube as well as stabilizes the generation of that plasma. Due to the use of the locally induced electric discharge and the electron cyclotron resonance for the generation and maintenance of the plasma, this radical generation device may also be called an ECR-LICP (Electron Cyclotron Resonance-Localized Inductively Coupled Plasma) type.

Various kinds of radicals can be used in radical-induced dissociation methods. It is preferable to selectively use radical species according to the purpose of the analysis, kind of compound and other factors. One possible method for selectively using various radical species in a single mass spectrometer is to change the kind of source gas supplied to the previously described type of radical generation device. For example, in the configuration adopted in a mass spectrometer disclosed in Patent Literature 4, either the hydroxyl and oxygen radicals or the hydrogen radical can be selectively introduced into the collision cell by switching the source gas between steam and hydrogen gas. Hydrogen radical is available not only for dissociating ions; due to its strong reducing effect, it can also act as a remover of an oxide film formed on the surface of an electrode within the collision cell by hydroxyl radical (or the like).

### CITATION LIST

### Patent Literature

Patent Literature 1: WO 2018/186286 A

Patent Literature 2: WO 2019/155725 A

Patent Literature 3: WO 2022/059247 A

Patent Literature 4: WO 2021/053865 A

### SUMMARY OF INVENTION

### Technical Problem

As described earlier, radical generation devices generate radicals from a source gas, such as steam or hydrogen gas. However, not all steam particles or hydrogen molecules in the source gas are changed to radicals; actually, a considerable amount of non-radical steam particles or hydrogen molecules are also introduced into the collision cell. Since steam particles and hydrogen molecules are highly reactive, there is the case where ions originating from a target sample react with not only radicals but also those non-radical steam particles or hydrogen molecules within the collision cell. The ease of occurrence of the latter reaction depends on the kind of sample and other related factors. If such a reaction of non-radical particles and ions occurs, it will be difficult to distinguish between the peaks of the ions resulting from the reaction with the non-radical particles and the peaks of the product ions resulting from the reaction with the radicals in the mass spectrum acquired through an MS/MS analysis employing radical-induced dissociation (this type of analysis is hereinafter called the “radical induction MS/MS analysis”). This causes problems in the task of analyzing the mass spectrum.

The present invention has been developed to solve the previously described problem. Its primary objective is to provide an ion analyzer and an ion analyzing method which enable a data analysis in which an ion generated by a reaction between an ion originating from a target sample and a component in a source gas from which a radical is generated is correctly distinguished from an ion generated by a reaction between the ion originating from the target sample and the radical.

### Solution to Problem

One mode of the ion analyzer according to the present invention is an ion analyzer which dissociates an ion originating from a sample by bringing a radical into contact with the ion within a reaction chamber, the ion analyzer including:
a generation chamber which communicates with the reaction chamber and into which a source gas from which the radical is to be generated is introduced;
a power supplier configured to supply electric power for generating an electric discharge within the generation chamber;
a source-gas supplier configured to supply the source gas to the generation chamber; and
a controller configured to control the source-gas supplier and the power supplier so as to perform, in a switchable manner, a radical introduction mode in which the supply of electric power to the generation chamber is performed while the source gas is supplied to the generation chamber for supplying the radical to the reaction chamber, and a source-gas-only introduction mode in which the supply of electric power to the generation chamber is not performed while the source gas is supplied to the generation chamber for supplying the source gas to the reaction chamber.

One mode of the ion analyzing method according to the present invention is an ion analyzing method which includes introducing a radical generated within a generation chamber into a reaction chamber and dissociating an ion originating from a sample by bringing the radical into contact with the ion within the reaction chamber, the ion analyzing method including:
a first step for performing an analysis under a condition under which a source gas from which the radical is to be generated can be changed to the radical and the radical can be supplied to the reaction chamber, by supplying the source gas to the generation chamber and supplying electric power for generating an electric discharge to the generation chamber; and
a second step for performing an analysis under a condition under which the source gas cannot be changed to the radical and the source gas can be supplied to the reaction chamber, by supplying the source gas to the generation chamber without supplying the electric power to the generation chamber.

### Advantageous Effects of Invention

In the previously described modes of the present invention, various kinds of gases can be used as the source gas. Typically, the source gas may be steam, hydrogen, oxygen, nitrogen or air. In the case of a mass spectrometer capable of a radical induction MS/MS analysis, the radical is used for dissociating an ion originating from a target sample. Therefore, according to common practice, a predetermined amount of electric power should be supplied to the generation chamber in order to generate a radical when the aforementioned kind of source gas is supplied to the generation chamber. By comparison, in the previously described modes of the present invention, an operation mode in which the source gas is directly introduced into the reaction chamber while the generation of radicals in the generation chamber is intentionally omitted can be selectively performed.

The previously described modes of the present invention enable the acquisition of mass spectrometry data under a condition under which an ion originating from a sample can react with non-radical particles (molecules) in the source gas without undergoing radical-induced dissociation. This type of mass spectrometry data cannot be acquired by conventional ion analyzers of this type. Therefore, for example, based on mass spectrometry data acquired for a sample without radical-induced dissociation and mass spectrometry data acquired for the same sample by a radical induction MS/MS analysis, an ion generated by a reaction between an ion originating from the sample and a component in the source gas can be correctly distinguished from an ion generated by a reaction between an ion originating from the sample and a radical, so that a structural analysis of a compound in the sample can be easily and accurately performed.

### BRIEF DESCRIPTION OF DRAWINGS

FIG. 1 is a schematic configuration diagram of a mass spectrometer as one embodiment of the present invention.

FIG. 2 is a configuration diagram of the main components of a radical generator in the mass spectrometer according to the present embodiment.

FIG. 3 is a functional block diagram of the data processing unit in FIG. 1.

FIG. 4 is a control flowchart for the process of acquiring m/z calibration information in the mass spectrometer according to the present embodiment.

FIG. 5 is a control flowchart for the process of performing a structural analysis in the mass spectrometer according to the present embodiment.

FIG. 6 is a control flowchart for the process of performing a gas-removing operation in the mass spectrometer according to the present embodiment.

### DESCRIPTION OF EMBODIMENTS

One embodiment of the ion analyzer and the ion analyzing method according to the present invention is hereinafter described with reference to the attached drawings. Although the following descriptions address a mass spectrometer as an embodiment of the ion analyzer, it is evident from the following descriptions that the present invention is not limited to this type of device but may also be applied in an ion mobility spectrometer or ion mobility-mass spectrometer.

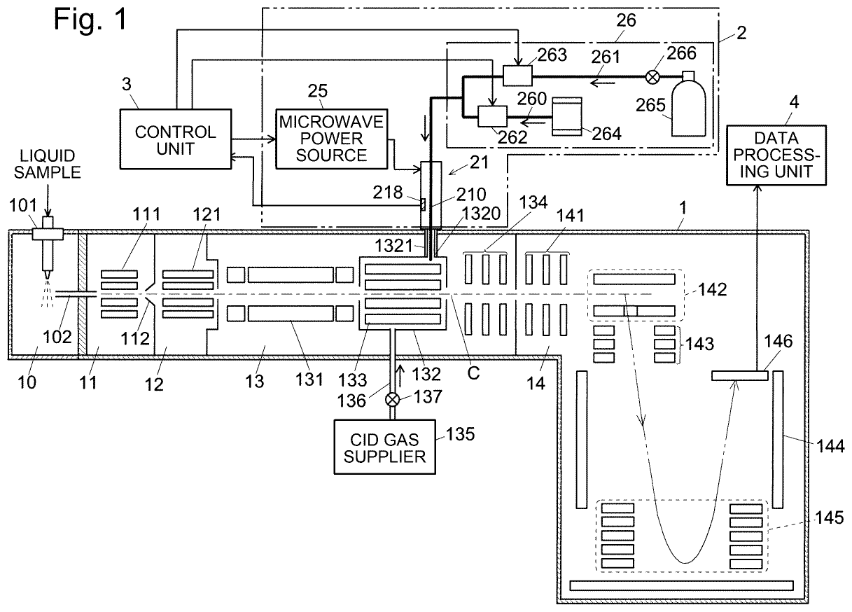

FIG. 1 is a schematic configuration diagram of the mass spectrometer according to the present embodiment. This mass spectrometer is a quadrupole time-of-flight (Q-TOF) mass spectrometer including an atmospheric pressure ion source.

As shown in FIG. 1, this mass spectrometer includes an ionization chamber 10 and a vacuum chamber 1. The inside of the ionization chamber 10 is at substantially atmospheric pressure. The inner space of the vacuum chamber 1 is divided into a plurality of sections (in the present embodiment, four chambers), which are a first intermediate vacuum chamber 11, second intermediate vacuum chamber 12, first analysis chamber 13 and second analysis chamber 14 sequentially arranged from the ionization chamber 10. Each of these chambers is evacuated by a vacuum pump (a rotary pump and/or a turbomolecular pump, not shown), forming the configuration of a multi-stage differential pumping system in which the degree of vacuum sequentially increases in the direction from the ionization chamber 10 toward the second analysis chamber 14.

The ionization chamber 10 is provided with an electrospray ionization (ESI) probe 101 configured to spray a liquid sample while imparting electric charges to the same sample. The ionization chamber 10 communicates with the first intermediate vacuum chamber 11 through a thin desolvation tube 102. The first intermediate vacuum chamber 11 is separated from the second intermediate vacuum chamber 12 by a skimmer 112 having a small hole at its apex. Ion guides 111 and 121 are located within the first and second intermediate vacuum chambers 11 and 12, respectively. Within the first analysis chamber 13, a quadrupole mass filter 131 configured to separate ions according to their m/z, a collision cell 132 having a multipole ion guide 133 inside, and an ion transport electrode 134 for transporting ions, are arranged along an ion beam axis C. The quadrupole mass filter 131 and the multipole ion guide 133 are each formed by a plurality of rod electrodes, while the ion transport electrode 134 is formed by a plurality of ring electrodes.

The collision cell 132 has an opening 1320 formed in its wall, with a cylindrical tube connection member 1321 provided so that one of its ends surrounds the opening 1320. A quartz tube 210 extending from a radical generation unit 2 is inserted into the tube connection member 1321. The end of the quartz tube 210 protrudes through the opening 1320 into the collision cell 132. Within the collision cell 132, an ion is dissociated by radicals supplied from the radical generation unit 2, as will be described later. A CID gas supply tube 136 for supplying a gas for collision induced dissociation (CID), which is typically argon gas, is connected to the collision cell 132. The other end of this tube is connected to a CID gas supplier 135 outside the vacuum chamber 1. A gas valve 137 for allowing/blocking a flow of gas is provided in the CID gas supply tube 136.

Within the second analysis chamber 14, the following components are provided: an ion transport electrode 141 for transporting ions coming from the first analysis chamber 13; an orthogonal accelerator 142 including a pair of electrodes facing each other across the ion beam axis C; an acceleration electrode 143; a flight tube 144 internally forming a flight space; a reflectron electrode 145 configured to form a return path for ions within the flight space; and an ion detector 146 configured to detect ions. For example, the ion detector 146 may be a multichannel plate detector. The detection signals produced by the ion detector 146 are sent to a data processing unit 4. A control unit 3 is configured to control the radical generation unit 2 and the data processing unit 4 as well as other components, including power sources for applying voltages to the related elements, although the signal lines required for the control are partially omitted in the drawing.

A typical radical induction MS/MS analysis operation performed under the control of the control unit 3 in the mass spectrometer configured in the previously described manner is as follows:

The ESI probe 101 receives a liquid sample supplied, for example, from the exit end of a column in a liquid chromatograph and sprays the liquid sample into the ionization chamber 10 while imparting electric charges to the same liquid sample, whereby a compound in the liquid sample is ionized. The generated ions are sent into the first intermediate vacuum chamber 11 through the desolvation tube 102. The ions which have entered the first intermediate vacuum chamber 11 are transferred through the ion guide 11, small hole of the skimmer 112 and ion guide 121 into the first analysis chamber 13, within which the ions are introduced into the quadrupole mass filter 131.

Among the various kinds of introduced ions, an ion having a specific m/z value is selectively allowed to pass through the quadrupole mass filter 131 and be introduced into the collision cell 132. Meanwhile, radicals are introduced from the radical generation unit 2 into the collision cell 132 through the quartz tube 210. The aforementioned ion introduced into the collision cell 132 is dissociated by reacting with the radicals. The various product ions resulting from the dissociation are extracted from the collision cell 132 and introduced into the orthogonal accelerator 142 through the ion transport electrodes 134 and 141.

The ions introduced into the orthogonal accelerator 142 along the ion beam axis Care ejected in a direction substantially orthogonal to the ion beam axis C at an appropriate timing. The ejected ions are accelerated by the acceleration electrode 143 and introduced into the flight space within the flight tube 144. The flying ions are repelled by the electric field created by the reflectron electrode 145 and ultimately reach the ion detector 146. The ion detector 146 produces a detection signal corresponding to the amount of incident ions. This signal is sent to the data processing unit 4. The period of time from the departure time of an ion from the orthogonal accelerator 142 to the arrival time of the ion at the ion detector 146, i.e., the time of flight of an ion, depends on the m/z value of that ion. Accordingly, based on the detection signals, the data processing unit 4 creates a time-of-flight spectrum showing the relationship between time of flight and ion intensity, and further creates a mass spectrum by converting the time of flight into the m/z value.

The present mass spectrometer allows for the use of a technique in which, in place of the radicals introduced from the radical generation unit 2 into the collision cell 132 through the quartz tube 210, CID gas is introduced into the collision cell 132 through the CID gas supply tube 136 so that an ion originating from a sample and introduced into the collision cell 132 is dissociated by CID. In that case, the various product ions resulting from the CID are extracted from the collision cell 132 and introduced into the orthogonal accelerator 142 through the ion transport electrodes 134 and 141, to be subjected to a mass spectrometric analysis as in the case of the previously described radical induction MS/MS analysis.

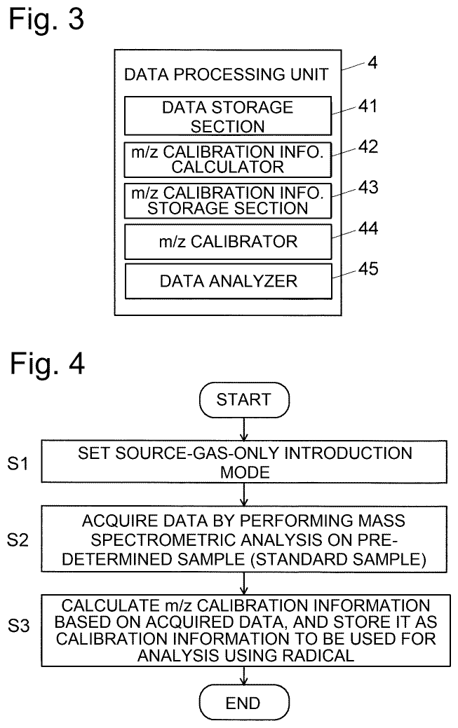

FIG. 3 is a block configuration diagram of the data processing unit 4 shown in FIG. 1. The data processing unit 4 includes a data storage section 41, m/z calibration information calculator 42, m/z calibration information storage section 43, m/z calibrator 44 and data analyzer 45 as its functional blocks. The function (operation) of each functional block will be described later.

The detailed configuration of the radical generation unit 2 and its operation are hereinafter described.

As shown in FIG. 1, the radical generation unit 2 includes a plasma generator 21. The plasma generator 21 generates a cloud of plasma from a source gas, such as steam, supplied from a source-gas supply source 26. The radicals generated in this plasma are supplied to the collision cell 132. A microwave power source 25 supplies the plasma generator 21 with microwave power for generating plasma.

The source-gas supply source 26 incudes a steam supply tube 260 provided with a first mass flow controller (MFC) 262, a hydrogen supply tube 261 provided with a second MFC 263, a water storage unit 264 connected to the inlet end of the steam supply tube 260, a hydrogen cylinder 265 connected to the inlet end of the hydrogen supply tube 261, and a manual on/off valve 266 provided in the hydrogen supply tube 261 between the second MFC 263 and the hydrogen cylinder 265. The steam and hydrogen supply tubes 260 and 261 have their remaining ends connected to the quartz tube 210. The water storage unit 264, which is equipped with a heater (not shown), can generate steam by heating the stored water and supply that steam through the steam supply tube 260. It should be noted that the hydrogen cylinder 265 and the manual on/off valve 266, which are parts of the source-gas supply source 26, are optional components which are added as needed and are not included in the mass spectrometer according to the present embodiment.

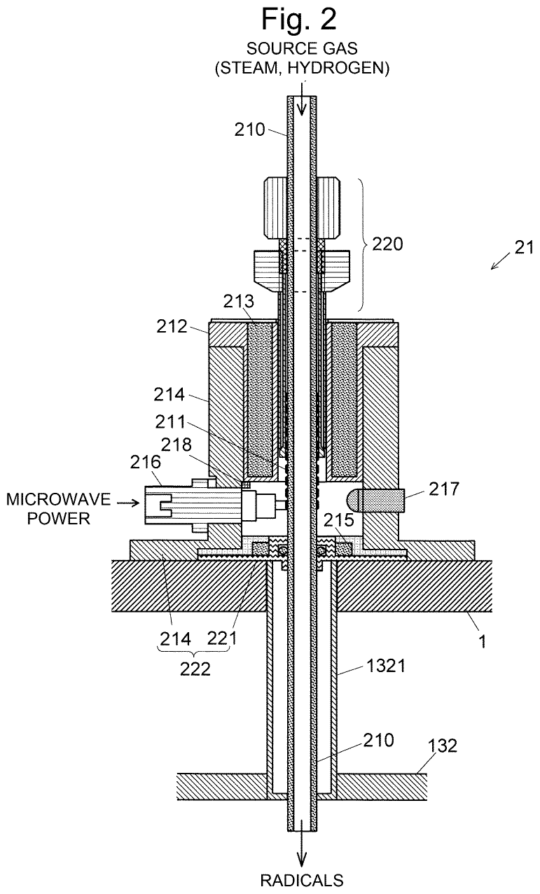

FIG. 2 is a vertical sectional view of the main components including the plasma generator 21.

The plasma generator 21 has the following components: a long, thin quartz tube 210; a helical antenna 211 consisting of a strip of conductor helically wound around the outer circumferential surface of a portion of the quartz tube 210; an outer conductive part 212 consisting of a conductor having a cylindrical cavity which is coaxial with the quartz tube 210 and has an inner diameter slightly larger than the outer diameter of the quartz tube 210; a permanent magnet 213 buried in the outer conductive part 212; a casing 214 holding the outer conductive part 212; and a permanent magnet 215 located at the bottom of the casing 214. The casing 214 is provided with a microwave supply connector 216, ultraviolet light source 217 and photodetector 218. When deep ultraviolet light is cast from the ultraviolet light source 217 onto the quartz tube 210, electrons are released from the wall surface of the quartz tube 210. Those electrons spark (ignite) the plasma.

The quartz tube 210 is a source-gas introduction tube for introducing a source gas from the source-gas supply source 26. Additionally, the inner space of this tube partially acts as a generation chamber, and partially as a radical passage. The microwave supply connector 216 is a coaxial connector to be connected to the microwave power source 25 via a coaxial cable. The conductor wire of the microwave supply connector 216 is connected to one end (in FIG. 2, the lower end) of the helical antenna 211. Though not shown, the outer conductive part 212 is grounded. A portion of the helical antenna 211 is electrically connected to the outer conductive part 212 via a resonator-tuning mechanism 220, and the helical antenna 211 is grounded at the point of that connection. The helical antenna 211, outer conductive part 212, resonator-tuning mechanism 220 and other related elements constitute a resonator for electron cyclotron resonance. The resonator-tuning mechanism 220, an example of which is disclosed in Patent Literature 3, is used for tuning the resonator.

This plasma generator 21 has a configuration called the “ECR-LICP” type which employs localized inductively coupled discharge and electron cyclotron resonance for the generation and maintenance of plasma.

On the bottom side of the casing 214, a substantially disk-shaped magnet holder 221 is attached. The casing 214 and the magnet holder 221 function as a holding member 222 for holding the quartz tube 210. The magnet holder 221 has an opening located in its central area through which the quartz tube 210 is to be inserted. As shown in FIG. 2, the plasma generator 21 is attached to the outer surface of the vacuum chamber 1, and the quartz tube 210 held by the holding member 222 is inserted into the tube connection member 1321 connecting the collision cell 132 and the vacuum chamber 1, reaching the inside of the collision cell 132. Since the inner space of the quartz tube 210 communicates with that of the collision cell 132, the inside of the quartz tube 210 will be in a vacuum state when no source gas is supplied to the quartz tube 210.

As described earlier, in the mass spectrometer according to the present embodiment, the source gas supplied from the source-gas supply source 26 passes through the quartz tube 210 and reaches the inside of the collision cell 132. In this process, the source gas is changed to radicals within an intermediate section of the line which corresponds to the generation chamber. The radicals thus generated are introduced into the collision cell 132 and contribute to the radical-induced dissociation.

The present mass spectrometer requires m/z calibration information for converting the time of flight into the m/z value in order to create a mass spectrum. This type of m/z calibration information is normally acquired based on the result of a measurement of a standard sample whose accurate m/z value is previously known. In the case of a conventional and common type of mass spectrometer which is capable of dissociating ions by CID and does not have a radical generation unit 2, the m/z calibration information is acquired by a mass spectrometric analysis in which a standard sample is introduced into the ionization chamber 10 while an appropriate amount of CID gas (argon) as the cooling gas is introduced into the collision cell 132 through the CID gas supply tube 136.

The m/z calibration information thus acquired may possibly be used for the m/z calibration in the radical induction MS/MS analysis. However, a study by the present inventor has revealed that the use of this information may cause a significant amount of mass discrepancy. In the case of the radical induction MS/MS analysis, since a source gas, such as steam or hydrogen gas, is introduced into the collision cell 132 in place of (or in some cases, in addition to) the CID gas, the state of gas within the collision cell 132 becomes different from when the CID gas is (solely) introduced. The “state of gas” in the present context means the gas concentration (density), size of gas molecules and other factors. Such a difference in the state of gas affects the kinetic energy and speed of the ion introduced into the collision cell 132. Therefore, the kinetic energy and speed which an ion possesses at the moment of the ejection of the ion in the orthogonal accelerator 142 will be different from when the CID gas is (solely) introduced. This is most likely to be the cause of the mass discrepancy.

To address this problem, the mass spectrometer according to the present embodiment is configured so that the m/z calibration information for a radical induction MS/MS analysis can be acquired beforehand and used for converting the time of flight into the m/z value in a time-of-flight spectrum acquired by a radical induction MS/MS analysis. For the acquisition of the m/z calibration information for a radical induction MS/MS analysis, a mass spectrometric analysis on a standard sample for calibration needs to be performed while the source gas is introduced from the radical generation unit 2 into the collision cell 132. The mass spectrometric analysis of the standard sample could be performed under a condition under which radicals can be generated in the radical generation unit 2. However, in that case, a sample that will be dissociated by radical-induced dissociation cannot be used, which limits the kinds of samples available as a standard sample. The mass spectrometer according to the present embodiment solves this problem by performing the mass spectrometric analysis on the standard sample using a source-gas-only introduction mode in which no microwave power is supplied to the helical antenna 211 while a source gas is supplied to the quartz tube 210 as in the radical induction MS/MS analysis.

An operation for acquiring the m/z calibration information for a radical induction MS/MS analysis in the mass spectrometer according to the present embodiment is hereinafter described. FIG. 4 is a control flowchart for this operation.

For example, when a command for initiating an operation for acquiring m/z calibration information has been issued through an input unit (not shown), the control unit 3 receives that command and sets the source-gas-only introduction mode as the operation mode (Step S1). In the source-gas-only introduction mode, the control unit 3 controls one or both of the MFCs 262 and 263 so that steam or hydrogen gas or both flow through the quartz tube 210 at predetermined gas flow rates. For example, the first MFC 262 may be controlled so that steam flows into the quartz tube 210 at a gas flow rate of 0.3 sccm. As another example, the MFCs 262 and 263 may be individually controlled so that steam and hydrogen gas flow into the quartz tube 210 at gas flow rates of 0.3 sccm and 0.2 sccm, respectively. Meanwhile, the control unit 3 does not energize the microwave power source 25. Therefore, the source gas with the flow rate regulated in the previously described manner flows through the quartz tube 210, to be directly introduced into the collision cell 132.

While the source gas is introduced into the collision cell 132 in the previously described manner, an appropriate kind of standard sample is electrostatically sprayed into the ionization chamber 10 from the ESI probe 101 or another ESI spray (not shown) dedicated for standard samples. The ions generated from this standard sample are guided through the ion guides 111 and 121, quadrupole mass filter 131, collision cell 132, and ion transport electrodes 134 and 141 into the orthogonal accelerator 142, to be ejected from this orthogonal accelerator 142 into the flight space within the flight tube 144 and be subjected to a mass spectrometric analysis. The signals obtained with the ion detector 146 in this analysis are converted into digital data and temporarily stored in the data storage section 41 (Step S2).

The m/z calibration information calculator 42 in the data processing unit 4 creates a time-of-flight spectrum based on the data stored in the data storage section 41 and calculates m/z calibration information for converting the time of flight into the m/z value, from a measured time of flight corresponding to the peak top of a peak located in the time-of-flight spectrum and the known (accurate) m/z value of the compound in the standard sample. It should be noted that the m/z calibration information does not always need to be information for converting the time of flight into the m/z value; for example, it may be the amount of discrepancy (a correction value of m/z) between the known (accurate) m/z value and the m/z value calculated from a measured time of flight based on a theoretical formula. The m/z calibration information thus calculated is stored in the m/z calibration information storage section 43 (Step S3).

The m/z calibration information stored in the m/z calibration information storage section 43 in this manner is used for calculating a mass spectrum from a time-of-flight spectrum acquired by a radical induction MS/MS analysis on any sample. That is to say, after a set of data has been acquired by a radical induction MS/MS analysis on a given sample, the m/z calibrator 44 in the data processing unit 4 converts the time of flight into the m/z value using the m/z calibration information stored in the m/z calibration information storage section 43 and creates a mass spectrum. The m/z calibration information used for this conversion has a high level of accuracy since it reflects the result of an analysis performed under substantially the same condition as applied when the radical induction MS/MS analysis was performed, except for the condition whether radicals are generated or not. Therefore, a mass spectrum with a high level of m/z accuracy can be obtained in a radical induction MS/MS analysis, so that a structural analysis of a compound can be accurately performed.

In a radical induction MS/MS analysis, when a plurality of kinds of gases, including steam, hydrogen gas, and a gas mixture of steam and hydrogen gas, are selectively used as the source gas, or when a different gas flow rate may possibly be selected, for example, for the same kind of gas species, it is preferable to previously acquire m/z calibration information for each of the different conditions. This further improves the accuracy of the m/z calibration information, so that the m/z accuracy of the mass spectrum in the radical induction MS/MS analysis can be further improved.

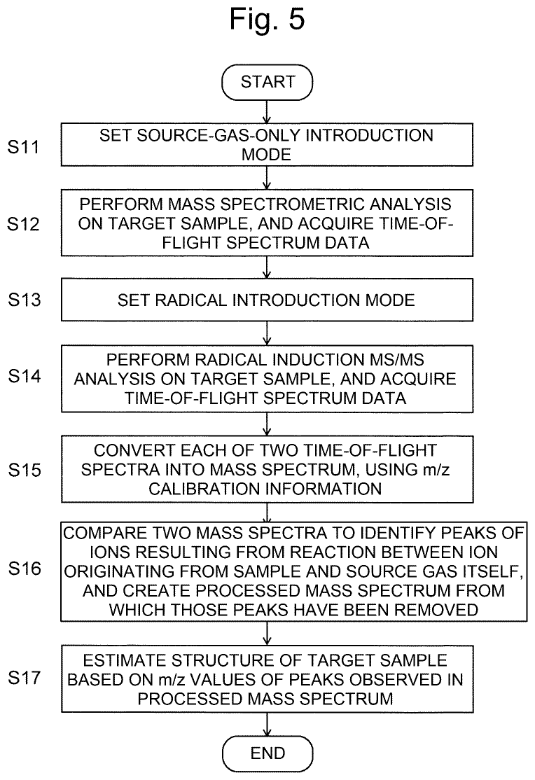

One example of the technique for data analysis using the previously described source-gas-only introduction mode in the mass spectrometer according to the present embodiment is hereinafter described. FIG. 5 is a control flowchart for performing this data analysis.

For example, when a command for initiating an analysis has been issued through an input unit (not shown), the control unit 3 receives that command and sets the source-gas-only introduction mode as the operation mode (Step S11). Similar to Step S1, in the source-gas-only introduction mode, the control unit 3 controls the MFCs 262 and 263 so that steam, hydrogen gas or a gas mixture of steam and hydrogen flows through the quartz tube 210 at a predetermined gas flow rate, while disabling the microwave power source 25. Therefore, the source gas with an appropriately regulated flow rate flows through the quartz 210, to be directly introduced into the collision cell 132.

A target sample is electrostatically sprayed from the ESI probe 101 into the ionization chamber 10. Ions generated from this target sample are introduced into the quadrupole mass filter 131 via the ion guides 111 and 121. The control unit 3 operates a power source (not shown) so that predetermined voltages are applied to the rod electrodes forming the quadrupole mass filter 131. Consequently, only an ion having a specific m/z, or ions falling within a specific range of m/z are selectively allowed to pass through the quadrupole mass filter 131 and guided through the collision cell 132 as well as ion transport electrodes 134 and 141 into the orthogonal accelerator 142, to be ejected from this orthogonal accelerator 142 into the flight space within the flight tube 144 and be subjected to a mass spectrometric analysis. The signals obtained with the ion detector 146 in this analysis are converted into digital data and temporarily stored in the data storage section 41 (Step S12). Since no radical is introduced into the collision cell 132, no radical-induced dissociation occurs within the collision cell 132.

Subsequently, the control unit 3 sets the radical introduction mode as the operation mode (Step S13). In the radical introduction mode, the control unit 3 controls the MFCs 262 and 263 in a similar manner to the source-gas-only introduction mode in Step S11, while energizing the microwave power source 25 to supply microwave power to the helical antenna 211. The microwave has, for example, a frequency of 2.5 GHz. Additionally, the control unit 3 energizes the ultraviolet light source 217 to initiate emission of deep ultraviolet light for helping the sparking of the plasma. Due to the microwave power supplied through the helical antenna 211 into the quartz tube 210, the source gas flowing within the quartz tube 210 is ionized, and the plasma ignites. The radicals generated within this plasma are introduced into the collision cell 132 along with the source gas.

A target sample identical to the analysis target in Step S12 is electrostatically sprayed from the ESI probe 101 into the ionization chamber 10. Ions generated from this target sample are introduced into the quadrupole mass filter 131 via the ion guides 111 and 121. The control unit 3 operates a power source (not shown) so that predetermined voltages are applied to the rod electrodes forming the quadrupole mass filter 131. Consequently, only an ion having a specific m/z, or ions falling within a specific range of m/z are selectively allowed to pass through the quadrupole mass filter 131 and be introduced into the collision cell 132. This time, since a large number of radicals are present within the collision cell 132, the ions originating from the target sample undergo radical-induced dissociation, whereby various product ions are generated. Those product ions are guided through the ion transport electrodes 134 and 141 into the orthogonal accelerator 142, to be ejected from this orthogonal accelerator 142 into the flight space within the flight tube 144 and be subjected to a mass spectrometric analysis. The signals obtained with the ion detector 146 in this analysis are converted into digital data and temporarily stored in the data storage section 41 (Step S14).

As a result of the processing of Steps S11-S14, the time-of-flight spectrum data under the source-gas-only introduction mode and the one under the radical introduction mode for the same target sample are stored in the data storage section 41. As described earlier, the time-of-flight spectrum data acquired under the radical introduction mode is a set of mass spectrometry data acquired by a radical induction MS/MS analysis. In the radical introduction mode, although the source gas is changed into radicals within the quartz tube 210, not all molecules contained in the source gas are changed into radicals; in practice, a portion of the molecules in the source gas become radicals, and the remaining molecules are introduced into the collision cell 132 in their original form. Steam and hydrogen gas are more active than the inert gas used as the CID gas (such as argon). Therefore, depending on the type of sample, the source gas and the ions originating from the sample possibly react with each other within the collision cell 132 and produce additional ions. The peaks formed by the ions generated in this manner may possibly appear in a mass spectrum along with the peaks of the product ions resulting from the radical-induced dissociation. In that case, it will be difficult to interpret the mass spectrum.

On the other hand, in the time-of-flight spectrum acquired under the source-gas-only introduction mode, no product-ion peak resulting from the radical-induced dissociation appears, while there are peaks corresponding to the ions resulting from the reaction of the source gas itself and the ions originating from the sample within the collision cell 132. Accordingly, by comparing the time-of-flight spectrum acquired under the radical introduction mode and the one acquired under the source-gas-only introduction mode, the product-ion peaks resulting from the radical-induced dissociation can be easily distinguished from the peaks formed by the ions resulting from the reaction between the ions originating from the sample and the source gas itself. Accordingly, the m/z calibrator 44 initially calculates a mass spectrum from each of the two time-of-flight spectra, using the m/z calibration information stored in the m/z calibration information storage section 43 (Step S15).

Then, the data analyzer 45 compares the two mass spectra to identify the peaks corresponding to the ions which have resulted from the reaction between the ion originating from the sample and the source gas itself. Since those peaks are unnecessary for the structural analysis, the data analyzer 45 removes those peaks to obtain a processed mass spectrum in which the product-ion peaks originating from the radical-induced dissociation mainly appear (Step S16). Furthermore, the data analyzer 45 estimates the structure of the compound in the target sample based on the m/z values of the peaks observed in the processed mass spectrum (Step S17).

In the previously described manner, in a radical induction MS/MS analysis, the influence of the ions resulting from the reaction between the ions originating from the sample and the source gas itself can be removed, so that a highly accurate structural analysis can be performed.

The task of distinguishing the peaks of the product ions generated by the radical-induced dissociation from those of the ions resulting from the reaction between the ions originating from the sample and the source gas itself may be performed in the stage of the time-of-flight spectrum before the conversion of the time-of-flight spectra into the mass spectra in Step S15. In that case, the mass spectrum can be acquired from a time-of-flight spectrum obtained by removing the latter group of peaks from the original time-of-flight spectra.

In the mass spectrometer according to the present embodiment, the steam vaporized from the water stored in the water storage unit 264 and hydrogen supplied from the hydrogen cylinder 265 (or hydrogen generator) attached to the end of the hydrogen supply tube 261 are used as the source gas. Air (external air) may possibly enter those tubes through some areas, such as a micro-sized opening in the MFC 262 or 263, water storage unit 264, hydrogen supply tube 261 or steam supply tube 260 while the present mass spectrometer is not used for a long period of time or when the inner space of the vacuum chamber 1 (or the like) in the device has been opened to the ambient air. Needless to say, air also enters a tube when the tube is reconnected.

The inside of the tubes located on the downstream side of the MFCs 262 and 263 (i.e., the side facing the plasma generation unit 2) is evacuated by the pump which evacuates the vacuum chamber 1. On the other hand, the air trapped within the tubes on the upstream side of the MFCs 262 and 263 (hydrogen supply tube 261 and steam supply tube 260) cannot be discharged to the outside as long as the MFCs 262 and 263 are not opened. If a radiofrequency discharge for creating radicals is induced through a source gas in which air is mixed, radicals of various gas species present in the air are generated, which may possibly contaminate some members located within the vacuum chamber 1. Therefore, it is preferable that no air is mixed in the source gas when the process of generating radicals from the source gas is to be initiated. To address this problem, the mass spectrometer according to the present embodiment is provided with a tube gas removal mode, as one of the operation modes, for forcibly removing air trapped within the hydrogen supply tube 261 and the steam supply tube 260.

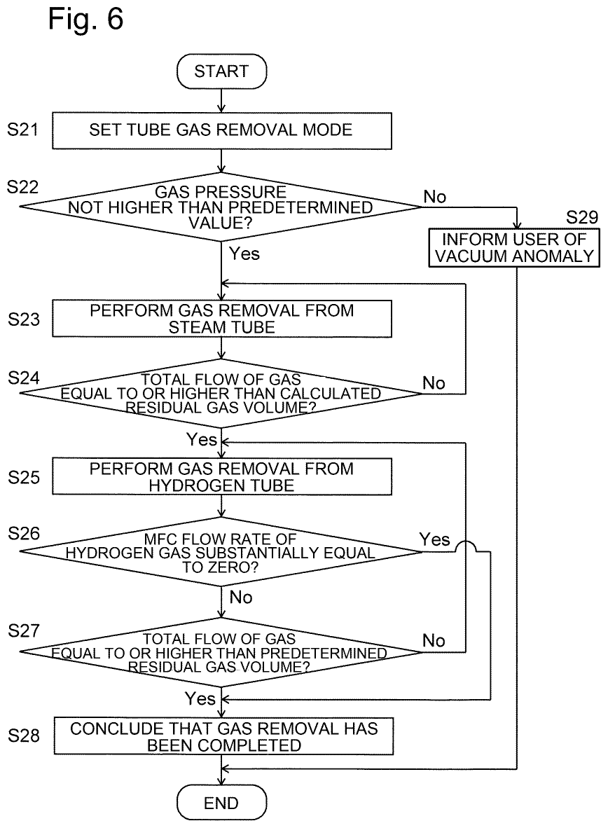

The tube gas removal mode is an operation mode in which the gas remaining in the water storage unit 264 as well as in the steam supply tube 260 and the hydrogen supply tube 261 on the upstream side of the MFCs 262 and 263 is removed by forcing the gas to flow into the evacuated side. FIG. 6 is a control flowchart of the tube gas removal mode in the mass spectrometer according to the present embodiment.

Before the tube gas removal mode is performed, the user closes the manual on/off valve 266 to block the supply of the hydrogen gas to the hydrogen supply tube 261. It should be noted that this operation is not essential; a case in which this operation is omitted is also taken into account, as will be described later.

For example, when a command for performing a tube-gas removal operation has been issued through an input unit (not shown), the control unit 3 receives this command and sets the tube gas removal mode as the operation mode (Step S21).

At the beginning of the tube gas removal mode, the control unit 3 initially detects the gas pressure, for example, within the first analysis chamber 13 with a vacuum gauge (not shown) to determine whether or not the gas pressure is not higher than a predetermined level (Step S22). If the gas pressure is higher than the predetermined level (i.e., if the degree of vacuum is low), there is the risk that allowing the gas within the tubes to flow into the vacuum chamber 1 would further lower the degree of vacuum and possibly cause failure due to the occurrence of an electric discharge between the electrodes (or other causes). Therefore, the gas pressure within the vacuum chamber 1 is checked before the execution of the tube gas removal, and if the gas pressure is higher than the predetermined level (“No” in Step S22), it is concluded that there is a vacuum anomaly. In that case, the control unit 3 discontinues the entire processing, showing a message on a display unit (not shown) to inform the user of the fact that the degree of vacuum is too low to perform the tube gas removal mode (Step S29).

On the other hand, when the gas pressure is not higher than the predetermined level, the control unit 3 performs the gas removal from the steam tube (Step S23). Specifically, the following processes are carried out.

The water storage unit 264 normally holds water. The steam vaporized from this held water is used as a source gas for generating radicals. A decrease in the amount of contained water causes a corresponding increase in the volume of the air space (the space above the water surface) within the water storage unit 264, which increases the maximum volume of the air that can flow into that space. Accordingly, the control unit 3 calculates the maximum volume of the air (“residual gas volume”) that can flow into the tube on the upstream side of the first MFC 262, including the water storage unit 264, based on the capacity of the water storage unit 264 and the inner volume of the tube of the steam supply tube 260 between the water storage unit 264 and the first MFC 262 (these have known, fixed values), as well as the initial volume of the water held in the water storage unit 264. The initial volume of the water held in the water storage unit 264 may be determined, for example, by assuming that the water is stored to the level of an initial level mark provided on the water storage unit 264. Based on the residual gas volume and the set value of the gas flow rate to be supplied through the first MFC 262, the control unit 3 calculates the period of time required for completely discharging the residual gas volume and allows the gas to be discharged to the downstream side though the first MDC 262 at least until the aforementioned period of time elapses, i.e., until the total volume of the flow of gas through the first MFC 262 becomes equal to or larger than the residual gas volume (Step S24). Thus, a condition in which no air is present within the water storage unit 264 and other related tubes can be created.

When the air within the tubes including the water storage unit 264 has been completely removed, the pressure within the water storage unit 264 becomes equal to the saturated vapor pressure. Accordingly, as opposed to the previously described case in which the point in time of the completion of the gas removal is determined from the calculated residual gas volume, a pressure gauge and a temperature sensor for measuring the pressure and the temperature within the air space of the water storage unit 264 may be installed, and whether or not the gas removal from the water storage unit 264 has been completed may be determined based on the measured pressure and a theoretical saturated vapor pressure at the measured temperature. A level sensor capable of detecting the level of the remaining water within the water storage unit 264 may additionally be provided for calculating the residual gas volume.

After the gas removal from the steam tube by Steps S23 and S24 has been completed, the control unit 3 subsequently performs the gas removal from the hydrogen gas tube (Step S25). Since the supply of the hydrogen gas is blocked as described earlier, a decrease in the amount of gas (air and hydrogen gas) within the hydrogen supply tube 261 on the upstream side of the second MFC 263 reduces the gas pressure within this tube, and ultimately, it will be impossible to force the gas to flow through the second MFC 263. Accordingly, the control unit 3 can determine whether or not the gas removal from the hydrogen gas tube has been completed by checking the flow rate of the actually flowing gas, which can be monitored at the second MFC 263. In other words, the control unit 3 continuously monitors this gas flow rate and concludes that the gas removal has been completed when the flow rate has been substantially zero (“Yes” in Step S26).

Since the length and tube diameter of the hydrogen supply tube 261 can be changed as needed by the user, the maximum amount of air that can flow into the tube is not fixed. Nevertheless, the previously described method for determining whether or not the gas removal has been completed ensures the removal of the entire amount of air regardless of the maximum amount of air that has flown into the tube. However, there is the possibility that the supply of hydrogen gas continues due to the user having forgotten to close the manual on/off valve 266, causing the determination result in Step S26 to be continuously “No”. Accordingly, even when the determination result in Step S26 is “No”, the control unit 3 conclusively determines that the gas removal has been completed when the total volume of the gas that has flown through the second MFC 263 has reached a preset value of the residual gas volume (“Yes” in Step S27) and completes the entire sequence of the gas-removing processes (Step S28). The preset value of the residual gas volume can be previously determined based on the maximum length and maximum diameter of a hydrogen gas tube commonly used (or recommended by the manufacturer of the device).

As described so far, the present device can carry out the tube gas removal mode in advance of the radical induction MS/MS analysis so as to remove gas from both the tube for steam and the one for hydrogen gas and thereby realize a state in which no air remains in those tubes. As noted earlier, if microwave power is supplied to generate radicals by inducing an electric discharge through steam or hydrogen gas in which (external) air is mixed, some problems may possibly occur, such as an internal contamination of the device or unsuccessful acquisition of appropriate measurement data. Those problems can be proactively prevented by executing the tube gas removal mode.

In normal cases, the gas flow rates of the MFCs 262 and 263 in the gas-removing process should preferably be approximately equal to the gas flow rates in the radical induction MS/MS analysis so as to avoid lowering the degree of vacuum within the vacuum chamber 1. However, if there is little concern regarding the deterioration of the degree of vacuum, as in the case where the evacuation power is extremely high, then the gas flow rates may be set to be higher than in the radical induction MS/MS analysis. This shortens the period of time required for the tube gas removal.

In order to more assuredly prevent the contamination of the device, it is preferable to always assume the maximum volume of the residual gas when performing the gas removal from the tubes including the water storage unit 264. However, that will require a considerably long period of time for the gas removal and impede an efficient analysis. Accordingly, for example, the gas removal on the assumption of the maximum volume of the residual gas may be performed only after the opening of the gas tubes to the ambient air has been detected, as in the process of pouring water into the water storage unit 264, while the gas removal with a previously determined fixed volume is performed in other situations, without calculating the volume of the residual gas.

It is also possible to previously assume the amount of leakage of air into the device during the period where the device is not in use (i.e., when the evacuation is not performed), and to estimate the residual gas volume based on the assumed value and the non-use period the device.

In the previously described tube gas removal mode, the gas removal from the steam tube including the water storage unit 264 and the gas removal from the hydrogen gas tube are sequentially performed. The device may be configured to allow these operations to be independently performed. More specifically, the device may allow for a selective execution of one of the gas removal modes according to a button operation (or the like). According to this configuration, for example, when the user has changed or reconnected a tube for hydrogen gas, the device can be operated to perform only the gas removal from the hydrogen gas tube, thereby avoiding an unnecessary gas-removing operation and improving the efficiency of the analyzing task.

In the previously described mass spectrometer, steam and hydrogen gas are used as the source gas from which radicals are to be generated. It is easy to conceive the idea that the present invention is also applicable in the case of using other gas species, such as nitrogen, as the source gas.

The previously described embodiment is an example in which the ion analyzer according to the present invention is applied in a Q-TOF mass spectrometer. It is evident that the present invention is not limited to the Q-TOF type. Specifically, the present invention is also applicable in other types of mass spectrometers in which an ion is dissociated for mass spectrometry, such as a triple quadrupole mass spectrometer, ion trap mass spectrometer or ion trap time-of-flight mass spectrometer.

The present invention is generally applicable in devices configured to dissociate an ion by using a radical generated in plasma and analyze product ions generated by the dissociation. Specifically, for example, the present invention is applicable in an ion mobility spectrometer configured to detect ions after separating them according to their ion mobility, as well as in an ion mobility-mass spectrometer configured to separate ions by using both ion mobility and m/z.

The configuration of the previously described mass spectrometer, and particularly that of the radical generation unit 2 for generating radicals, is a mere example and can naturally be appropriately changed or modified to any configuration that can generate radicals by using plasma. The radicals in the present context include hydroxyl radical, hydrogen radical, oxygen radical, nitrogen radical and other kinds of radicals commonly used for radical-induced dissociation methods, as well as various kinds of molecules and atoms which are in an excited state due to externally imparted energy or in a metastable state.

The previously described embodiment and various modifications are mere examples of the present invention. It is evident that any change, modification, addition or the like appropriately made within the spirit of the present invention will also fall within the scope of claims of the present application.

### Various Modes

A person skilled in the art can understand that the previously described illustrative embodiment is a specific example of the following modes of the present invention.

(Clause 1) One mode of the ion analyzer according to the present invention is an ion analyzer which dissociates an ion originating from a sample by bringing a radical into contact with the ion within a reaction chamber, the ion analyzer including:
a generation chamber which communicates with the reaction chamber and into which a source gas from which the radical is to be generated is introduced;

a power supplier configured to supply electric power for generating an electric discharge within the generation chamber;
a source-gas supplier configured to supply the source gas to the generation chamber; and
a controller configured to control the source-gas supplier and the power supplier so as to perform, in a switchable manner, a radical introduction mode in which the supply of electric power to the generation chamber is performed while the source gas is supplied to the generation chamber for supplying the radical to the reaction chamber, and a source-gas-only introduction mode in which the supply of electric power to the generation chamber is not performed while the source gas is supplied to the generation chamber for supplying the source gas to the reaction chamber.

(Clause 6) One mode of the ion analyzing method according to the present invention is an ion analyzing method which includes introducing a radical generated within a generation chamber into a reaction chamber and dissociating an ion originating from a sample by bringing the radical into contact with the ion within the reaction chamber, the ion analyzing method including:
a first step for performing an analysis under a condition under which a source gas from which the radical is to be generated can be changed to the radical and the radical can be supplied to the reaction chamber, by supplying the source gas to the generation chamber and supplying electric power for generating an electric discharge to the generation chamber; and
a second step for performing an analysis under a condition under which the source gas cannot be changed to the radical and the source gas can be supplied to the reaction chamber, by supplying the source gas to the generation chamber without supplying the electric power to the generation chamber.

In the ion analyzer according to Clause 1 and the ion analyzing method according to Clause 6, an operation mode in which the source gas is directly introduced into the reaction chamber while the generation of radicals in the generation chamber is intentionally omitted can be selectively performed. This enables the acquisition of mass spectrometry data under a condition under which an ion originating from a sample can react with non-radical particles (molecules) in the source gas without undergoing radical-induced dissociation. This type of mass spectrometry data cannot be acquired by conventional ion analyzers of this type. Therefore, by the ion analyzer according to Clause 1 and the ion analyzing method according to Clause 6, for example, based on mass spectrometry data acquired for a sample without radical-induced dissociation and mass spectrometry data acquired for the same sample by a radical induction MS/MS analysis, an ion generated by a reaction between an ion originating from the sample and a component in the source gas can be correctly distinguished from an ion generated by a reaction between an ion originating from the sample and a radical, so that a structural analysis of a compound in the sample can be easily and accurately performed.

Furthermore, by the ion analyzer according to Clause 1 and the ion analyzing method according to Clause 6, mass spectrometry data on an appropriate standard sample for mass calibration can be acquired by performing a mass spectrometric analysis on that standard sample while introducing a source gas into a reaction chamber in a substantially similar manner to a radical generation process, i.e., under the condition that the source gas is present within the reaction chamber. Based on this mass spectrometry data, highly accurate mass calibration information that can be used for a radical induction MS/MS analysis can be acquired, and a highly accurate mass spectrum can be created from mass spectrometry data acquired by a radical induction MS/MS analysis.

(Clause 2) The ion analyzer according to Clause 1 may further include a data analyzer configured to perform a data analysis of a compound in a sample based on mass spectrometry data acquired for the sample under the radical introduction mode and mass spectrometry data acquired for the same sample under the source-gas-only introduction mode.

(Clause 7) The ion analyzing method according to Clause 6 may further include a data-analyzing step for performing a data analysis of a compound in a sample based on mass spectrometry data acquired for the sample in the first step and mass spectrometry data acquired for the same sample in the second step.

By the ion analyzer according to Clause 2 and the ion analyzing method according to Clause 7, product ions resulting from a reaction between an ion originating from a sample and a radical can be correctly identified in a mass spectrum acquired by a radical induction MS/MS analysis, by removing, from the mass spectrum, the peaks of ions which have resulted from a reaction between an ion in the sample and a component in the source gas and are therefore unnecessary (obstructive) for the data analysis. Therefore, a structural analysis of a compound in a sample can be easily and accurately performed.

(Clause 3) The ion analyzer according to Clause 1 or 2 may further include a calibration information calculator configured to calculate mass calibration information based on mass spectrometry data acquired for a predetermined sample under the source-gas-only introduction mode.

(Clause 4) The ion analyzer according to Clause 3 may further include a calibrator configured to calibrate mass information in the mass spectrometry data acquired under the source-gas-only introduction mode, using the mass calibration information.

(Clause 8) The ion analyzing method according to Clause 6 or 7 may further include a calibration information calculation step for calculating mass calibration information based on mass spectrometry data acquired for a predetermined sample in the second step.

(Clause 9) The ion analyzing method according to Clause 8 may further include a calibration execution step for calibrating mass information in the mass spectrometry data acquired in the first step, using the mass calibration information.

The ion analyzers according to Clauses 3 and 4 as well as the ion analyzing methods according to Clauses 8 and 9 can acquire mass calibration information using a standard sample while the source gas is introduced into a reaction chamber in a substantially similar manner to a radical generation process. Accordingly, even a sample which may possibly undergo radical-induced dissociation can be used as a standard sample. The thereby acquired information is not mass calibration information acquired while argon or similar gas used as a CID gas or cooling gas is introduced into the reaction chamber, but mass calibration information acquired while a source gas is introduced into the reaction chamber. The mass calibration information thus acquired almost correctly reflects the mass discrepancy of mass spectrum data acquired by a radical induction MS/MS analysis. Therefore, a mass spectrum with a high level of mass accuracy can be created, and the accuracy of the structural analysis based on that mass analysis can also be improved.

(Clause 5) In the ion analyzer according to one of Clauses 1-4, the source-gas supplier may include a gas regulator configured to regulate the flow rate of a flow of the source gas supplied to a gas passage connected to the generation chamber or allow/block the flow of the source gas to the gas passage, and
the controller may be configured to control the gas regulator so as to perform a tube gas removal mode in which the flow of the source gas is supplied to the gas passage in the gas regulator until a predetermined condition is satisfied.

The ion analyzer according to Clause 5 can remove unwanted gas remaining in a tube, such as (external) air, from the tube by performing the tube gas removal mode in advance of the execution of a radical induction MS/MS analysis. If radicals are generated when air which contains various impurities remains within a tube, the radicals originating from those impurities will be introduced into the device and possibly contaminate an electrode or other related elements. To address this problem, the ion analyzer according to Clause 5 can prevent radicals originating from impurities from being introduced into the device. This reduces the amount of wasted time for the cleaning of the device or other tasks, and also enables the collection of satisfactory measurement data with little or no contamination of the electrodes or other elements.

### REFERENCE SIGNS LIST

1 . . . Vacuum Chamber
10 . . . Ionization Chamber
11 . . . First Intermediate Vacuum Chamber
12 . . . Second Intermediate Vacuum Chamber
13 . . . First Analysis Chamber
14 . . . Second Analysis Chamber
101 . . . ESI Probe
102 . . . Desolvation Tube
111, 121 . . . Ion Guide
112 . . . Skimmer
131 . . . Quadrupole Mass Filter
132 . . . Collision Cell
1320 . . . Opening
1321 . . . Tube Connection Member
133 . . . Multipole Ion Guide
134, 141 . . . Ion Transport Electrode
135 . . . CID Gas Supplier
136 . . . CID Gas Supply Tube
137 . . . Gas Valve
142 . . . Orthogonal Accelerator
143 . . . Acceleration Electrode
144 . . . Flight Tube
145 . . . Reflectron Electrode
146 . . . Ion Detector
2 . . . Radical Generation Unit
21 . . . Plasma Generator
210 . . . Quartz Tube
211 . . . Helical Antenna
212 . . . Outer Conductive Part
213 . . . Permanent Magnet
214 . . . Casing
215 . . . Permanent Magnet
216 . . . Microwave Supply Connector
217 . . . Ultraviolet Light Source
218 . . . Photodetector
220 . . . Resonator-Tuning Mechanism
221 . . . Magnet Holder
222 . . . Holding Member
25 . . . Microwave Power Source
26 . . . Source-Gas Supply Source
260 . . . Steam Supply Tube
261 . . . Hydrogen Supply Tube
262, 263 . . . Mass Flow Controller (MFC)
264 . . . Water Storage Unit
265 . . . Hydrogen Cylinder
266 . . . Manual On/Off Valve
3 . . . Control Unit
4 . . . Data Processing Unit
41 . . . Data Storage Section
42 . . . m/z Calibration Information Calculator
43 . . . m/z Calibration Information Storage Section
44 . . . m/z Calibrator
45 . . . Data Analyzer
C . . . Ion Beam Axis

1. An ion analyzer which dissociates an ion originating from a sample by bringing a radical into contact with the ion within a reaction chamber, the ion analyzer comprising:
a generation chamber which communicates with the reaction chamber and into which a source gas from which the radical is to be generated is introduced;
a power supplier configured to supply electric power for generating an electric discharge within the generation chamber;
a source-gas supplier configured to supply the source gas to the generation chamber; and
a controller configured to control the source-gas supplier and the power supplier so as to perform, in a switchable manner, a radical introduction mode in which a supply of electric power to the generation chamber is performed while the source gas is supplied to the generation chamber for supplying the radical to the reaction chamber, and a source-gas-only introduction mode in which the supply of electric power to the generation chamber is not performed while the source gas is supplied to the generation chamber for supplying the source gas to the reaction chamber.

2. The ion analyzer according to claim 1, further comprising a data analyzer configured to perform a data analysis of a compound in a sample based on mass spectrometry data acquired for the sample under the radical introduction mode and mass spectrometry data acquired for the same sample under the source-gas-only introduction mode.

3. The ion analyzer according to claim 1, further comprising a calibration information calculator configured to calculate mass calibration information based on mass spectrometry data acquired for a predetermined sample under the source-gas-only introduction mode.

4. The ion analyzer according to claim 3, further comprising a calibrator configured to calibrate mass information in the mass spectrometry data acquired under the source-gas-only introduction mode, using the mass calibration information.

5. The ion analyzer according to claim 1, wherein:
the source-gas supplier includes a gas regulator configured to regulate a flow rate of a flow of the source gas supplied to a gas passage connected to the generation chamber or allow/block the flow of the source gas to the gas passage, and
the controller is configured to control the gas regulator so as to perform a tube gas removal mode in which the flow of the source gas is supplied to the gas passage in the gas regulator until a predetermined condition is satisfied.

6. An ion analyzing method which includes introducing a radical generated within a generation chamber into a reaction chamber and dissociating an ion originating from a sample by bringing the radical into contact with the ion within the reaction chamber, the ion analyzing method comprising:
a first step for performing an analysis under a condition under which a source gas from which the radical is to be generated can be changed to the radical and the radical can be supplied to the reaction chamber, by supplying the source gas to the generation chamber and supplying electric power for generating an electric discharge to the generation chamber; and
a second step for performing an analysis under a condition under which the source gas cannot be changed to the radical and the source gas can be supplied to the reaction chamber, by supplying the source gas to the generation chamber without supplying the electric power to the generation chamber.

7. The ion analyzing method according to claim 6, further comprising a data-analyzing step for performing a data analysis of a compound in a sample based on mass spectrometry data acquired for the sample in the first step and mass spectrometry data acquired for the same sample in the second step.

8. The ion analyzing method according to claim 6, further comprising a calibration information calculation step for calculating mass calibration information based on mass spectrometry data acquired for a predetermined sample in the second step.

9. The ion analyzing method according to claim 8, further comprising calibration execution step for calibrating mass information in the mass spectrometry data acquired in the first step, using the mass calibration information.
